(19) United States
(12) Patent Application Publication
(10) Pub. No.: US 2024/0173371 A1
(43) Pub. Date: May 30, 2024

(54) RHODIOLA CRENULATA COMPOSITIONS AND METHODS OF MAKING THE SAME

(71) Applicant: NutriScience Innovations LLC, Milford, CT (US)

(72) Inventors: Michael Lelah, Arlington Heights, IL (US); David Bom, Huntley, IL (US)

(21) Appl. No.: 18/515,601

(22) Filed: Nov. 21, 2023

Related U.S. Application Data
(60) Provisional application No. 63/428,842, filed on Nov. 30, 2022.

(51) Int. Cl. A61K 36/41; A61K 31/7032

(52) U.S. Cl. CPC A61K 36/41; A61K 31/7032; A61K 2236/333; A61K 2236/51

(57) ABSTRACT

The present disclosure relates generally to dietary supplements and compositions containing Rhodiola crenulata administered in therapeutically effective amounts for effecting health in a mammal. Disclosed are compositions containing an improved extract of Rhodiola crenulata containing a higher content level of salidrosides (>5%), and a reduced number of or absence of other flavonoids. In this manner, the resulting novel composition has enhanced bioactivity of salidroside, and therefore, improved treatment and health benefit possibilities.

### TECHNICAL FIELD

The present disclosure relates generally to dietary supplement compositions and methods of making the compositions utilizing extracts of Rhodiola crenulata. The present disclosure relates to improved dietary supplement compositions including Rhodiola crenulata and various modified extracts thereof, having synergistic effects for use as treatments of various ailments and conditions, as well as to provide various health benefits. These treatments and benefits may include antioxidant effects and reductions in stress and/or fatigue.

### BACKGROUND

The Rhodiola genus comprises more than 200 species of plants, which are naturally found in various regions including Tibet, China, Mongolia, the Himalayan region, Europe and North America. Rhodiola plants are used in traditional medicines in Asia. Traditional uses of Rhodiola include tonics, adaptogens, antidepressant, anti-inflammatory, altitude sickness and fatigue/stress relief products to name a few. Rhodiola rosea (Goldenroot) is the most common medicinal Rhodiola species. Rhodiola rosea contains salidroside (also described as salidrosides) and rosavin (also described as rosavins). Salidroside, which is believed to be the more bioactive of the two compounds, is a glycosylated analog of tyrosol, while the rosavins are a series of mono and disaccharides of cinnamyl alcohol.

Rhodiola crenulata (also described as crenulate) is another Rhodiola species. It is unusual because, unlike Rhodiola rosea and many other Rhodiola species, it does not contain rosavins. In addition, the compositions of the present disclosure utilize an extract composition with content levels of salidrosides elevated to 6% versus the typical 1-3%.

Rhodiola has known health benefits. For example, Rhodiola extracts are referred to as adaptogens. Adaptogens are known to function to normalize physiologic responses to stress, thereby enhancing work performance and increasing stress tolerance. Rhodiola extracts have been observed to reduce the release of cortisol in response to stress, which plays a role in its antifatigue effects.

Various known studies have found that extracts of the Rhodiola plant were effective for providing certain health benefits, including extending lifespan, anti-aging activity, and having antioxidant and anti-inflammatory activity. For example, in a study conducted in fruit flies (Drosophila melanogaster) it was observed that the lifespan of the flies was increased.¹ It was proposed that this effect was due to an increase in the resistance of organs toward stress, while also reducing oxidative stress. In a rat model it was found that the antioxidant activity of a rhodiola plant extract successfully reduced lipid peroxidation.² In an in vitro study an extract from Rhodiola rosea inhibited the activities of COX-1, COX-2 and PLA-2. It was noted that the PLA-2 activity was the most likely source of Rhodiola's anti-inflammatory activity.³ In a separate publication it was reported that an extract of Rhodiola rosea inhibited inflammatory c-reactive protein and creatinine kinase expression after heavy exercise.⁴

In connection with potential benefits to the immune system and antidepressive effects of Rhodiola extract, a study on Wistar male rats determined that supplementation with salidroside increased both the total CD3+T cells and the CD4+T helper cells.⁵ Rhodiola extract was found to reduce depressive symptoms in patients with mild to moderate depression.⁶ Oral delivery of salidroside to rats at 20 and 40 mg/kg over the course of two weeks alleviated olfactory-bulbectomy-induced hyperactivity and improved the outcome of a forced swim test.⁷ Rhodiola rosea versus Rhodiola crenulata

The biological effects of Rhodiola rosea will differ from Rhodiola crenulata since the latter contains higher levels of salidrosides and does not contain rosavins. Salidroside is believed to be the major component that contributes to the favorable health benefits produced by the plant. Almost pure salidroside has been synthesized using synthetic biology. The Rhodiola plant contains about 1% salidrosides and other flavonoids. Commercial natural extracts of Rhodiola typically contain 1-3% salidrosides. Rhodiola crenulata used in the present disclosure similarly does not contain rosavins, and therefore contains higher content levels of the more bioactive salidroside. Thus, the Rhodiola crenulata of the present disclosure provides unique compositions and improved bioactivity not seen in Rhodiola crenulata and traditional Rhodiola rosea.

There is a need for a novel extract of Rhodiola crenulata which contains a higher level of salidrosides (>5%), together with a reduction of, or near elimination of, most of the other flavonoids present in the extract. The present disclosure is not simply purification of the extract, but a targeted reduction of, and/or near elimination of, many of the other flavonoids from the extract, thereby creating a unique composition. Reduction or elimination of other flavonoids allows for the resulting extract to contain bioactive salidrosides, which have known health benefits, while eliminating many of those other flavonoids that may interfere with the bioactivity of the salidrosides. Thus, eliminating or nearly eliminating other flavonoids not only increases the percentage of salidrosides, but also is desired for enhancing the activity of the extract.

A need, therefore, exists for improved dietary supplement compositions of extracts of Rhodiola crenulata with higher content levels of salidroside and the reduction of and/or near elimination or elimination of many other flavonoids.

A need further exists for methods of making the dietary supplement compositions providing synergistically effective extracts of Rhodiola crenulata, wherein the process includes modifications to the extracts, modifications to the biomass, and combinations of the extracts with other herbals and dietary ingredients.

A need further exists for an improved, novel extract of Rhodiola crenulata containing a higher content level of salidrosides (>5%), together with a targeted reduction or near elimination of many other flavonoids.

A need further exists for an improved, novel extract of Rhodiola crenulata containing a higher content level of salidrosides (>5%) without many other flavonoids, wherein the composition has higher salidroside bioactivity.

### SUMMARY

The present disclosure relates generally to dietary supplement compositions and methods of making the compositions utilizing extracts of Rhodiola crenulata. The present disclosure includes improved dietary supplement compositions including Rhodiola crenulata and various modified extracts thereof, having synergistic effects for use as treatments of various ailments and conditions and to provide various health benefits.

The present disclosure provides a composition of Rhodiola crenulata containing a higher content level of salidrosides (>5%), wherein the salidrosides have enhanced bioactivity.

In one embodiment, the present disclosure provides a composition containing an improved, novel extract of Rhodiola crenulata containing a higher content level of salidrosides (>5%) and a reduced number of many other flavonoids.

In another embodiment, the present disclosure provides a composition containing an improved, novel extract of Rhodiola crenulata containing a higher content level of salidrosides (>5%) and a targeted elimination of many other flavonoids.

In yet another embodiment, the present disclosure provides a method of extracting Rhodiola crenulata to increase the content level of salidrosides and to reduce or eliminate the number of many other flavonoids.

Another embodiment the present disclosure provides a method of treating an extract of Rhodiola crenulata by increasing the pH level of the extraction solvent with a solvent media of modified pH. This method is designed to increase the content level of salidosides, while reducing and/or eliminating many other targeted flavonoids in the resulting extract.

In yet another embodiment, the present disclosure provides a method of treating an extract of Rhodiola crenulata by increasing the pH level of the extract by post processing or further processing the extract with a solvent media of modified pH. This embodiment increases the content level of salidosides, while reducing and/or targeting the elimination or near elimination of many other flavonoids in the resulting extract. Post-processing refers to starting with the Rhodiola crenulata extract and dissolving the extract in solvent media, which is distinct from an initial extraction where raw Rhodiola crenulata is extracted in the solvent media.

It is another advantage and objective of the present disclosure to provide an improved dietary supplement composition including extracts of Rhodiola crenulata having a synergistic effect on anti-aging, antioxidant, antidepression and anti-inflammatory activities and treatments.

### BRIEF DESCRIPTION OF THE DRAWINGS

FIG. 1 illustrates an HPLC chromatogram of extractions of Rhodiola crenulata with different pH adjustment between pH 3.5 to pH 8.5. Acid adjustment at pH 3.5 is shown for both post-processing (chromatogram 1) and first extraction adjustment (chromatogram 4). pH adjustment to pH 8.5 is shown in chromatogram 2 where almost no flavonoids remain in the latter run-time region;

FIG. 2 illustrates an HPLC chromatogram of 6% extracts of Rhodiola crenulata without pH adjustment according to Example 5;

FIG. 3 illustrates an HPLC chromatogram of water/ethanol extract of Rhodiola crenulata with pH adjustment to pH 8.5 according to Example 5;

FIG. 4 illustrates an HPLC chromatogram of water/ethanol extract of Rhodiola rosea according to Example 5.

### DETAILED DESCRIPTION

The present disclosure generally relates to dietary supplements and compositions containing Rhodiola crenulata extracts administered in therapeutically effective amounts for effecting health in a mammal. Disclosed are compositions containing an improved extract of Rhodiola crenulata containing a higher content level of salidrosides (>5%), and a reduced number of, or absence of, many other flavonoids. In this manner, the resulting composition has enhanced bioactivity, and therefore, improved treatment and health benefit possibilities.

The present disclosure also provides methods of creating unique compositions of Rhodiola crenulata extracts. Notably, the present disclosure is not simply purification of the extract, but a targeted reduction of, and/or near elimination of, many of the other flavonoids from the extract, thereby resulting in a unique composition containing primarily bioactive salidrosides, Eliminating or nearly eliminating other flavonoids through the present method both increases the percentage of salidrosides, and also enhances the activity of the resulting extract.

Based on this unusual characteristic of containing salidrosides and not rosavins, the present disclosure proposes to further modify the extracts to create unique and novel dietary supplement compositions. These process modifications may be: (a) modifications to the extraction process (typically in the liquid state prior to drying the extract); (b) modifications to the biomass (including fermentation and/or enzymatic treatment; and (c) combinations of the extract or its modifications in (a) and (b) with other herbals and dietary ingredients.

### Post-Processing of the Rhodiola crenulata Extract

When the Rhodiola crenulata extract (1 g) is suspended in water (15 ml) it forms a turbid, opaque, dark reddish-brown suspension with a pH of about 4.9. Adjustment of the pH of the liquid extract may be achieved through the addition of an acid selected from L-malic acid, hydrochloric acid, citric acid, ascorbic acid, acidic resins or from a base selected from potassium hydroxide, sodium hydroxide, potassium carbonate, potash, sodium bicarbonate, ammonium hydroxide or basic resins, or other natural acids or alkalis. Ambient or thermal treatment of the extract may include utilization of a metal catalyst including but not limited to iron, copper, zinc, palladium or platinum. Ambient or thermal treatment of the extract may include clay such as bentonite alone or in combination with a metal. Ambient or thermal treatment of the extract may also include activated charcoal to deodorize and/or reduce color. Ambient or thermal treatment of the extract may include treatment with air or oxygen in combination with a metal catalyst and/or clay.

The following are examples of the various pH modifications to the extract and resulting unique compositions:

### EXAMPLE 1—NEUTRALIZATION (pH 7.0) POST-PROCESSING

Rhodiola crenulata extract (1 g) was suspended in 15 ml of water and treated with 5 drops of 1M potassium hydroxide (KOH) from a plastic 0.5 ml disposable transfer pipet. The initial pH of the mixture was 4.9. After the KOH addition the pH was 7.2. After stirring for 2 h, the pH had equilibrated to 7.0. The solution was reddish-brown and turbid prior to and after KOH addition. The contents were frozen and lyophilized providing 0.939 g of a reddish-brown solid product.

### EXAMPLE 2—SLIGHTLY BASIC (pH 7.9) POST PROCESSING

Rhodiola crenulata extract (1 g) was suspended in 15 ml of water and treated with 10 drops of 1M KOH from a plastic 0.5 ml disposable transfer pipet. The initial pH of the mixture was 4.9. After the KOH addition the pH was 8.1. After stirring for 2 h, the pH had equilibrated to 7.9. The solution was reddish-brown and turbid prior to and after KOH addition. The contents were frozen and lyophilized providing 0.915 g of a reddish-brown solid product.

### EXAMPLE 3—MORE BASIC (pH 9.1) POST-PROCESSING

Rhodiola crenulata extract (1 g) was suspended in 15 ml of water and treated with 15 drops of 1M KOH from a plastic 0.5 ml disposable transfer pipet. The initial pH of the mixture was 4.9. After the KOH addition the pH was 9.0. After stirring for 2 h, the pH had equilibrated to 9.1. The solution was reddish-brown and turbid prior to KOH addition, but it remained dark after pH adjustment, but the turbidity went away. The contents were frozen and lyophilized providing 0.998 g of a reddish-brown solid product.

### EXAMPLE 4

In a series of water/ethanol (40/60 ratio) extractions of Rhodiola crenulata root with different pH adjustment of the extraction media to between pH 3.5 to pH 8.5, the fluids after extraction were analyzed by HPLC (High Performance Liquid Chromatography). The method of HPLC analysis is similar to that described in the USP for powdered Rhodiola rosea extracts section on Composition.

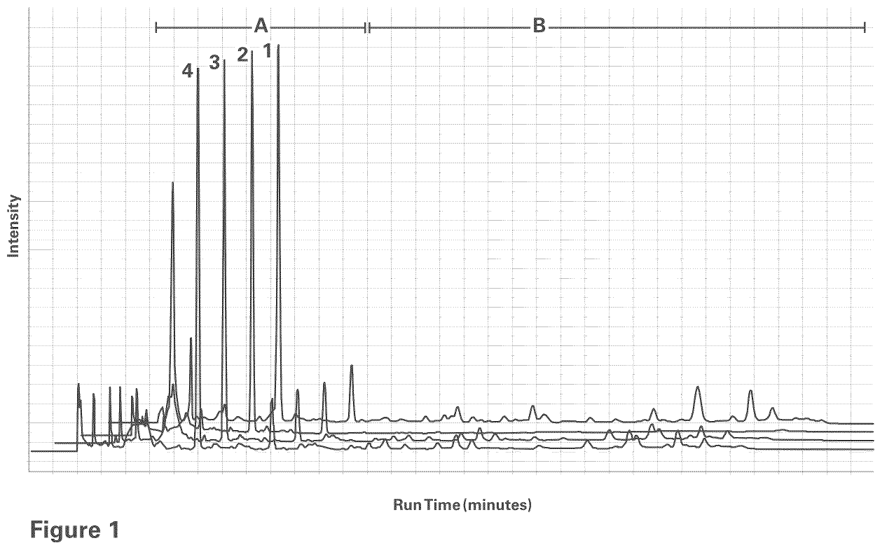

As shown in FIG. 1, the natural pH of the extract solution is about pH 5.0. The primary peaks 1-4 (region A) are the salidrosides peak, with a number of smaller peaks at later run times (region B). Specifically, the peaks are defined as: Peak 1—Chromatogram with pH adjusted to 3.5 (post-processing); Peak 2—Chromatogram with pH adjusted to 8.5. Almost no flavonoids remain; Peak 3—Chromatogram with no pH adjustment; Peak 4—Chromatogram with pH adjusted to 3.5. The smaller peaks at later run times in region B are other flavonoids. Run time (or retention time) is determined by the interaction of the flavonoid with the stationary phase in the HPLC column. The stronger the interaction, the longer the run time. The smaller peaks at longer times are thus peaks of flavonoids which have specific chemistries with stronger interactions with the stationary phase in the HPLC column.

With acid adjustment, either in the extraction media or in post processing the extract, no significant changes in the smaller peaks at longer runs times occur. With alkali adjustment to pH 8.5 either in the extraction media or in post processing the extract, the smaller peaks at longer run times mostly and significantly disappear, indicating a unique composition of Rhodiola crenulata extract has been obtained with an almost complete elimination of certain flavonoids, which are identified by longer run times.

### EXAMPLE 5

Extraction with water and ethanol (40/60 ratio), following the methods described in Examples 4 for a 6% salidrosides extract was conducted on Rhodiola crenulata root, with a pH adjustment of the extraction media to pH 8.5 using potassium hydroxide. The resultant extract was dried and then analyzed.

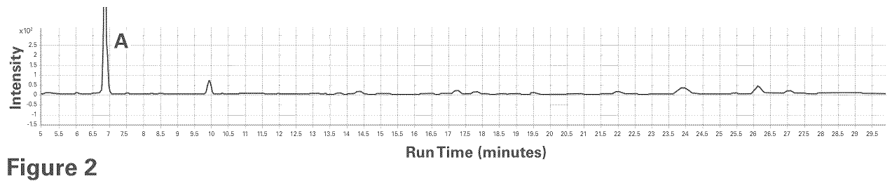

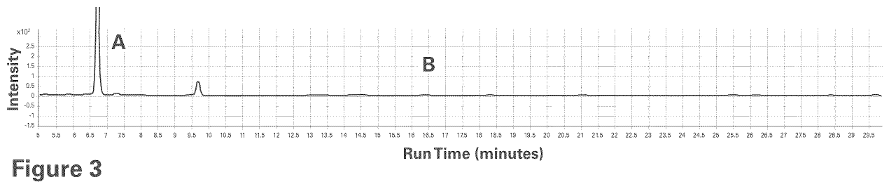

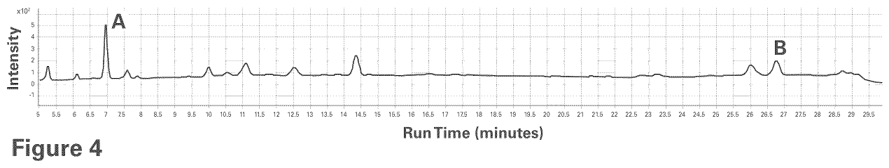

HPLC chromatographs for the extract in Example 5 are shown in FIGS. 2-4. Included for comparison are 6% extracts of Rhodiola crenulata without the pH adjustment (FIG. 2), a 3% extract of Rhodiola crenulata without the pH adjustment (FIG. 3), and a water/ethanol extract of Rhodiola rosea (FIG. 4). The salidrosides peak is at about run time 6.5 to 7 minutes. The rosavins peak is at about run time 26.8 minutes. From the chromatograms, the Rhodiola rosea extract contains rosavins, as is well known. None of the Rhodiola crenulata extracts contain any rosavins.

In particular, the water/ethanol extract of Rhodiola crenulata with pH adjustment to pH 8.5, is identified as being unique by chromatographic peaks only between the range of approximately 5 and 11 minutes run time (FIG. 3 region A). There are almost no peaks in the run time range 11 through 30 minutes (FIG. 3 region B). This specific set of peaks and more importantly, an absence of peaks, uniquely defines a novel composition of Rhodiola crenulata with pH 8.5 adjustment and differentiates it from a standard extract of Rhodiola crenulate root and from a standard extract of Rhodiola rosea (FIG. 4), both of which have significant peaks between 11 and 30 minutes run time. In addition, as shown in FIG. 4, Rhodiola rosea has a specific peak of rosavins (region B) at about 26.8 minutes, which is not present in Rhodiola crenulata.

### REFERENCES

¹Jafari, M. et al., Rhodiola: A Promising Anti-aging Chinese Herb., Rejuvenation Res., 2007, 10, p. 587.
²Gupta, A., et al., Effects of Rhodiola imbricata on Dermal Wound Healing., Planta Letter 2006, p. 1.
³Bawa, AS., et al., Anti-inflammatory activity of Rhodiola rosea-“A Second Generation Adaptogen”., Phytother. Res., 2009, 23, p. 1099.
⁴Abidov, M. et al., Extract of Rhodiola rosea radix reduces the level of c-reactive protein and creatinine kinase in the blood. Bull. Exp. Biol. Med., 2004, 138, p. 63.
⁵Lu, L. et al., Rejuvenating activity of salidroside (SDS): Dietary intake of SDS enhances the immune response of aged rats., Age, 2013, 35, p. 637.
⁶Darbinyan, V. et al., Clinical Trial of Rhodiola rosea L. extract SHR-5 in the treatment of mild to moderate depression., Nord J. Psychiatry 2007, 61, p. 343.
⁷Yang SJ, et al., Antidepressant-like effects of salidroside on olfactory bulbectomy-induced pro-inflammatory cytokine production and hyperactivity of HPA axis in rats., Pharmacol. Biochemi. Behav. 2014, 124, p. 451.

1. A dietary supplement composition comprising an extract of Rhodiola crenulata containing a content level of salidrosides greater than 5 percent.

2. The dietary supplement composition of claim 1, wherein the composition contains a reduced amount of other flavonoids.

3. The dietary supplement composition of claim 1 wherein the composition includes an elevated activity level of the salidrosides.

4. The dietary supplement composition of claim 3, wherein the composition having the elevated activity level of salidrosides with a reduced amount of other flavonoids is used as to provide a health benefit including stress reduction, an antioxidant effect, or other health benefit.

5. A method of producing an extract of Rhodiola crenulata, the method including the steps of;
providing an extraction fluid having a pH between 7.1 and 11;
adding Rhodiola crenulata root into the extraction fluid; and,
drying the extraction fluid into an extraction powder.

6. The method of claim 5, wherein the method further includes the step of determining a content level of flavonoids in the extraction powder.

7. The method of claim 6, wherein a content level of salidrosides is determined by HPLC chromatography.

8. The method of claim 6, wherein the content level of other flavonoids is determined by HPLC chromatography.

9. The method of claim 5, wherein the method further includes the step of eliminating or nearly eliminating an amount of other flavonoids in the extraction powder.

10. The method of claim 5, wherein the extraction fluid is adjusted to between pH 8 to pH 9 using a base or alkali.

11. The method of claim 5 where the extraction fluid or fluids is selected from the group comprising water, ethanol or other alcohol, water/ethanol or other alcohol combination, or supercritical carbon dioxide.

12. The method of claim 5, wherein the method includes providing a water/ethanol extract of Rhodiola crenulata with a pH of the extraction fluid adjusted to pH 8.5 using potassium hydroxide or other alkali.

13. A method of producing an extract of Rhodiola crenulata, the method including the steps of;
providing a first extraction fluid;
adding Rhodiola crenulata root into the extraction fluid;
drying the extraction fluid into a first extraction powder;
adding the first extraction powder to a second extraction fluid having a pH between 7.1 and 11; and,
drying the extraction fluid into a second extraction powder.

14. The method of claim 13, wherein the method further includes determining a content level of flavonoids in the second extraction powder.

15. The method of claim 14, wherein the method further includes the step of reducing an amount of other flavonoids in the second extraction powder.

16. The method of claim 14, wherein the method further includes the step of eliminating or nearly eliminating an amount of other flavonoids in the second extraction powder.

17. The method of claim 14, wherein content level of salidrosides in the second extraction powder is determined by HPLC chromatography.

18. The method of claim 16, wherein the content level of other flavonoids in the second extraction powder is determined by HPLC chromatography.
